(19) United States
(12) Patent Application Publication
(10) Pub. No.: US 2008/0077001 A1
(43) Pub. Date: Mar. 27, 2008

(54) Medical information system for intensive care unit

(75) Inventors: Richard Ruscio, Spencerport, NY (US); David H. Foos, Rochester, NY (US)

Correspondence Address: Pamela R. Crocker;Patent Legal Staff, Eastman Kodak Company, 343 State Street, Rochester, NY, 14650-2201, US

(73) Assignee: Eastman Kodak Company

(21) Appl. No.: 11/506,342

(22) Filed: Aug. 18, 2006

(51) Int. Cl. A61B 5/05

(52) U.S. Cl. 600/407

(57) ABSTRACT

A method for longitudinal tracking of a patient in a critical care facility. A first diagnostic image at a time t1 is obtained, taken using a first set of imaging parameters. At least a portion of the first set of imaging parameters is stored. A second diagnostic image is obtained at a time t2, later than time t1, using a second set of imaging parameters. At least a portion of the second set of imaging parameters is stored. First and second diagnostic images are of substantially the same body tissue. A region of interest is identified from either the first or second diagnostic image. A computer aided diagnostic process executes for a portion of the region of interest on each of the first and second diagnostic images. Results of the computer aided diagnostic process are compared.

### FIELD OF THE INVENTION

This invention generally relates to medical devices and related information systems, and more particularly relates to a system providing clinical imaging information for a patient in an intensive care unit.

### BACKGROUND OF THE INVENTION

The DICOM (Digital Imaging and Communications in Medicine) standard was developed in order to manage the potentially large amounts of patient data that are available from a range of diagnostic and imaging systems. Developed and maintained as a joint effort through the National Electrical Manufacturers Association (NEMA), the DICOM data interchange standard has the goal of providing a common framework for acquisition, transmission, archival, retrieval, and presentation of medical images and related patient data from a variety of imaging modalities and environments. Benefits from DICOM conformance include interoperability of equipment from different manufacturers so that patient data, once obtained, can be accessible for display, printing, diagnostic assessment, and storage, without requiring proprietary systems and software. For example, DICOM conformance can allow images from any of a number of different types of equipment to be processed on a single Computer-Aided Diagnosis (CAD) system. Results from the CAD system can then be stored and used for viewing or presentation by other conforming systems.

The DICOM standard itself is sizable, defining data structures, communication protocols, and interaction models for data transfer between systems. The DICOM Structured Report (SR) provides a standard data structure for allowing medical data and images, obtained at a number of different types of equipment from a number of different vendors, to be more readily accessible and usable by a clinician. Various DICOM functions have been implemented as providers of medical hardware and software work to allow more effective integration of patient information. However, there are still shortcomings in implementation that prevent the full benefits of this standardization from being available to those who care for patients.

One area of particular interest relates to DICOM support for patients in an Intensive Care Unit (ICU) or similar type of critical care facility. Patients generally remain in an Intensive Care Unit (ICU) for a brief but active period of time. The activities typically carried out in the ICU include the following: Electronic monitoring of vital signs; Periodic or event-driven visits and data collection; Medication and device delivery; Radiographic and other imaging studies; and ICU activities can be planned and routine or, in many cases, unexpected.

In some instances, one or more treatments may be administered in a repeated cycle including (i) administering treatment and (ii) measuring response, until a successful outcome is achieved.

A typical patient's stay in the ICU is characterized by a considerable amount of monitoring, both short and longer term, as one or another interventions are performed in order to improve the patient's condition. Timing can be critical, certainly more than is typical in other care wards. Critical incidents can occur with some frequency, sometimes as consequence of a sequence of activities. Typically, there is generally a great deal of activity in an ICU, with multiple event cycles that may overlap, without synchronization.

The need for accurate, up-to-date information on each ICU patient can be of primary importance in this environment. Recognizing how critical this problem can be, various vendors of medical information systems and apparatus offer solutions that are particularly directed at the ICU and critical care environment. As one example, Picis Inc., Wakefield, Mass., markets an information system designed to integrate documentation from other systems that store patient information. Similarly, GE Healthcare Systems, Waukesha, Wis., offer Centricity Critical Care Clinisoft as a dedicated information management system for critical care facilities.

FIG. 1 shows a conventional ICU information system 101 that provides patient records information for an ICU patient that has been obtained from a facility Electronic Patient Records (EPR) system 120 and from ICU logging apparatus 130. The patient EPR information is labeled 140. ICU events 150 that may be recorded include, for example, data collected by the ICU Staff, which describe active interventions with the patient, and are entered into an available computer system. These events could include observations, delivery of medications, changes of intravenous solutions, and non-clinical nursing interventions, for example. Some type of data accumulation function performed by a data accumulator 160 obtains this information and makes it available as data 170 for retrieval and use by an attending clinician, such as on a report viewing station 190.

While such conventional solutions help to make patient information and medical history more readily available, some significant shortcomings remain. One area of concern relates to image processing. Conventional systems allow data accumulation to a single control console or display, but provide no tools for correlation of the data or for use of image data obtained from different imaging modalities (such as from x-rays and ultrasound, for example) or obtained at different times.

One shortcoming of conventional systems relates to a lack of tools that help a clinician in tracking a condition. For example, while conventional systems may make earlier-obtained information available to a physician, nurse, or technician, little attention is paid to chronological order, which provides added dimension and meaning to measured data, as is well appreciated by those skilled in the medical arts.

A shortcoming of existing ICU information systems solutions relates to the use of chronological relationships between or among images. This applies both to images obtained earlier in the patient's history and those obtained during the period of ICU treatment. Existing system solutions do not take advantages of the benefits that can be obtained from chronological sequencing, correlation of image data, and automated methods for analyzing images taken at different times. As one example, multiple radiological or ultrasound images, taken under similar parameters, may be obtained over short periods of time to detect an excess of fluid in a patient's lungs. For such a condition, rates of change over time can be particularly useful data; however, conventional systems fail to correlate or coordinate image information obtained at different times. The clinician does the work of arranging and correlating a succession of images in order to track the progress of such a condition.

Thus, there is a need for improved management, use, and presentation of patient metadata, imaging metadata, measurement data, and images obtained at different times for improving the level of patient care in ICU and other critical care environments.

### SUMMARY OF THE INVENTION

According to one aspect of the present invention, there is provided a method for longitudinal tracking of a patient in a critical care facility. The method includes: a) obtaining a first diagnostic image at a time t1, taken using a first set of imaging parameters; b) storing at least a portion of the first set of imaging parameters; c) obtaining a second diagnostic image at a time t2, time t2 being later than time t1, taken using a second set of imaging parameters, wherein the first and second diagnostic images are of substantially the same body tissue; d) storing at least a portion of the second set of imaging parameters; e) identifying a region of interest from either the first or second diagnostic image; f) executing a computer aided diagnostic process for a portion of the region of interest for each of the first and second diagnostic images to generate first and second image results; and g) comparing the first and second image results.

The present invention can provide a system solution to the problem of patient image and information management for ICU and other care facilities. The present invention integrates patient data from a variety of sources, including past and present image data, electronic patient records, and ICU nursing log data.

An advantage of the present invention is that it enables the use of CAD capabilities for image diagnosis in an ICU environment.

These and other objects, features, and advantages of the present invention will become apparent to those skilled in the art upon a reading of the following detailed description when taken in conjunction with the drawings wherein there is shown and described an illustrative embodiment of the invention.

### BRIEF DESCRIPTION OF THE DRAWINGS

The foregoing and other objects, features, and advantages of the invention will be apparent from the following more particular description of the embodiments of the invention, as illustrated in the accompanying drawings. The elements of the drawings are not necessarily to scale relative to each other.

FIG. 1 is a block diagram showing the scope of existing solutions for ICU patient data management and display.

FIG. 2 is a block diagram showing components of an ICU patient data management system according to the present invention.

FIG. 3 is a block diagram showing processes and data for image processing according to the present invention.

FIG. 4 is a block diagram outlining the overall structure of an ICU Structured Record for a patient according to one embodiment.

FIG. 5 is a block diagram showing the processes and data for providing a chronologically arranged information set according to one embodiment.

FIG. 6 is a block diagram showing part of the overall process for displaying chronologically arranged image data.

FIG. 7 is a plan view of an example display showing how chronologically arranged image data can be displayed to a clinician.

FIG. 8 is a plan view of an example display showing another arrangement of chronologically arranged images and data.

### DETAILED DESCRIPTION OF THE INVENTION

The present description is directed in particular to elements forming part of, or cooperating more directly with, apparatus in accordance with the invention. It is to be understood that elements not specifically shown or described may take various forms well known to those skilled in the art.

The methods and apparatus of the present invention are directed to supporting improved patient care in an ICU or similar critical care environment. As noted earlier in the background section, these environments are characterized by substantial demands on staff and urgency of attention to patient needs. To the extent possible, the present invention utilizes and extends existing DICOM data structures and protocol in order to provide enhanced opportunities for patient care in the ICU.

The general term “metadata” as used in the present application is used broadly and can include any of a number of types of data that support the diagnostic image data or measurement data for a patient, exclusive of the actual image data itself that stores pixel values or of the measured values themselves. Loosely defined as “data about data”, metadata for an image, or imaging metadata, typically includes data obtained about image capture conditions, devices, settings, and other data having some relation to an image and the conditions under which it was obtained. Methods used in the present invention employ any/all available data about the patient, whether or not it could be considered as metadata or as other data under any applied definition. Since the patient is the subject of interest, metadata that supports an image or that supports measurement data could include patient identification and history information or a logical link to this information, using this understanding of the term. Metadata could include, for example, text data or data encoded in some other fashion that indicates how image densities can be interpreted. Text data could be stored, for example, in ASCII format or some other conventional format, including any of a number of compressed data formats. Metadata can also support measurement data values, such as those obtained from various medical instruments used to measure vital signs of the patient, for example.

The concept of diagnostic image comparison for what has been termed as “longitudinal tracking” has been proposed for specific types of medical images, such as described in U.S. Patent Application No. 2005/0251021 entitled “Methods and Systems for Generating a Lung Report” by Kaufman et al. which relates to tracking of lung nodules from images obtained at different times. U.S. Patent Application No. 2005/0010106 entitled “Methods for the Compensation of Imaging Technique in the Processing of Radiographic Images” by Lang et al. describes a type of “longitudinal” tracking that uses comparison of earlier vs. later images for monitoring osteoporosis and other conditions in bone tissue. The need for longitudinal tracking, both of images and of measured data, is heightened for a patient in an ICU or similar critical care environment. For example, it is noted that, unlike the cases described in the Kaufman et al. patent application and Lang et al. patent application, two or more ICU patient imaging sessions can be performed within a few days, or even more than once a day, sometimes only a few hours apart. In addition to frequency of obtaining images, there can be a need to use measured data more effectively, since measured data can also be employed for longitudinal tracking in many cases.

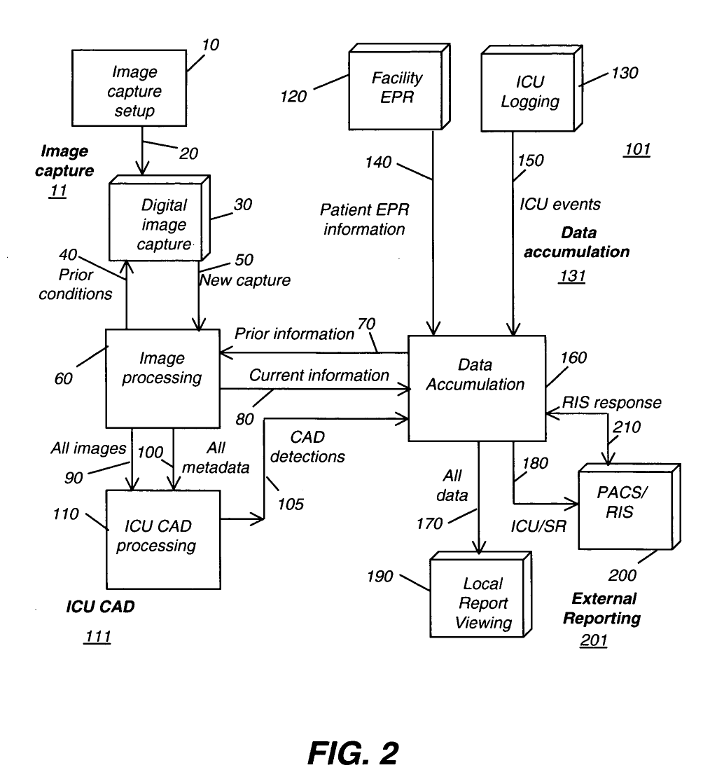

Thus, patients in an ICU can be subject to multiple, frequent examinations, including procedures that obtain measured data and images. Referring to FIG. 2, there is shown a system block diagram of an information system 101 showing functional units of a system apparatus and information elements that make use of patient images and measured data according to one embodiment of the present invention. Four sections of the system include: an image capture section 11, an ICU CAD section 111, a data accumulation section 131, and an external reporting section 201.

Initially, a clinician orders an imaging study, for example, from the radiology practice group, by means of a Radiology Information System (RIS). When the order is executed, a radiology technologist carries out the procedure for diagnostic image capture. The order, along with radiology procedures and practices, defines an image capture set-up instruction 10. A first image taken for this patient requires initial conditions 20 to be set and recorded as image metadata. A digital image capture process 30 is carried out and generates a new capture 50. Subsequent images taken for identical radiology studies for the patient, then require the use of prior conditions 40. Those prior conditions, as diagnostic imaging metadata, can be used in conjunction with the image capture setup and initial condition information, to provide the optimal setup conditions for subsequent image captures.

When the capture is completed, an image processing process 60 occurs. Image processing process 60 uses new capture 50, as well as the conditions of the current and prior captures, referred to as prior information 70, to generate the final image captured. The accumulation of all prior and latest information and images, referred to as current information 80, becomes part of the accumulated data set for this patient.

An ICU CAD process 110 uses the available information to perform its analysis. Its available information set includes all images 90 and the associated metadata 100 and other data for the patient. It is again emphasized that metadata 100 is interpreted broadly and can include any data related to the image or measurement data obtained for a patient. Output from ICU CAD process 110, CAD detections 105, become part of the accumulated data set for this patient. ICU CAD process 10 applies CAD analysis to image data obtained while the patient is in the ICU, along with other image data available for the patient from earlier imaging sessions if longitudinal tracking is needed. CAD utilities and techniques are well known to those skilled in the medical imaging arts and include capabilities for detection of tissue abnormalities based on intensity data, gradient data, texture analysis, shape detection, and other utilities. Some representative types of CAD capabilities used for mammography, lung cancer detection, and other types of diagnostic imaging are described, for example, in U.S. Pat. No. 6,748,044 entitled “Computer Assisted Analysis of Tomographic Mammography Data” to Sabol et al.; in U.S. Patent Application No. 2004/0247166 entitled “Method, System, and Computer Readable Medium for an Intelligent Search Workstation For Computer Assisted Interpretation of Medical Images” by Giger et al.; and in U.S. Pat. No. 4,907,156 entitled “Method and System for Enhancement and Detection of Abnormal Anatomic Regions in a Digital Image” to Doi et al. Use of DICOM structures and protocols for storage and transmission of CAD results is described in U.S. Pat. No. 6,909,795 entitled “Communicating Computer-Aided Detection Results in a Standards-Based Medical Imaging Environment” to Tecotzky et al. Generally, CAD results need not be stored for earlier images, since CAD utilities can be executed on images obtained at the most recent time t1 or at earlier times t2, t3, . . . tn.

Electronic Patient Records (EPR) in EPR system 120 for a medical facility include, for a hospital, the repository of clinical and administrative information that is stored (or is otherwise accessible) at the facility for the patient. Patient EPR Information 140 is used as an input to ICU CAD process 110 as well as for presentation to clinicians and radiologists. Of special interest is demographic information, medical history, and current clinical measurements, as well as other types of patient metadata.

ICU Events 150 include data collected by the ICU Staff, which describe active interventions with the patient, and are entered into an available computer system. The events of this type include, but are not limited to, observations, delivery of medications, changes of intravenous solutions, and non-clinical nursing interventions, for example.

Considerable data available about the patient, such as current imaging information, EPR information, ICU events, and patient images from each imaging session, remain available to the system for retrieval and use, as part of the function of a data accumulator 160. Data accumulator 160 itself can be a function performed on a dedicated computer workstation or may be one of a number of functions that operate either on a single computer or on a distributed computer system.

Depending upon the information technology (IT) tools available at the facility using the system of the present invention, those sources of data may or may not be capable of alerting data accumulator 160 that new information is available. Where automatic reporting is not done, data accumulator 160 should be able to poll those information sources periodically in order to maintain a state of currency.

There are at least two specified output purposes for the accumulated data.

The first use, local report viewing 190, provides a visual, time-line oriented, view of data 170 that is available for the patient. The data can include projection radiographs; image metadata that describes the image capture conditions; CAD indications obtained from analysis of the image data on a CAD system; EPR data from the hospital facility; and ICU Log information obtained from the ICU staff during treatment, for example. The second use is to create appropriate records, that will be stored into a PACS/RIS (Picture Archiving and Communications System/Radiological Information System) system 200. The first of these records, Intensive Care Unit Structured Record (ICU/SR) 180, is intended to allow some or all of the information available in the ICU to be directly available to the radiologist via PACS. The second record, RIS Response 210, is the storage of image and metadata required to begin completion of the work initiated by the processing of the initial RIS radiology order, by the radiology technologist.

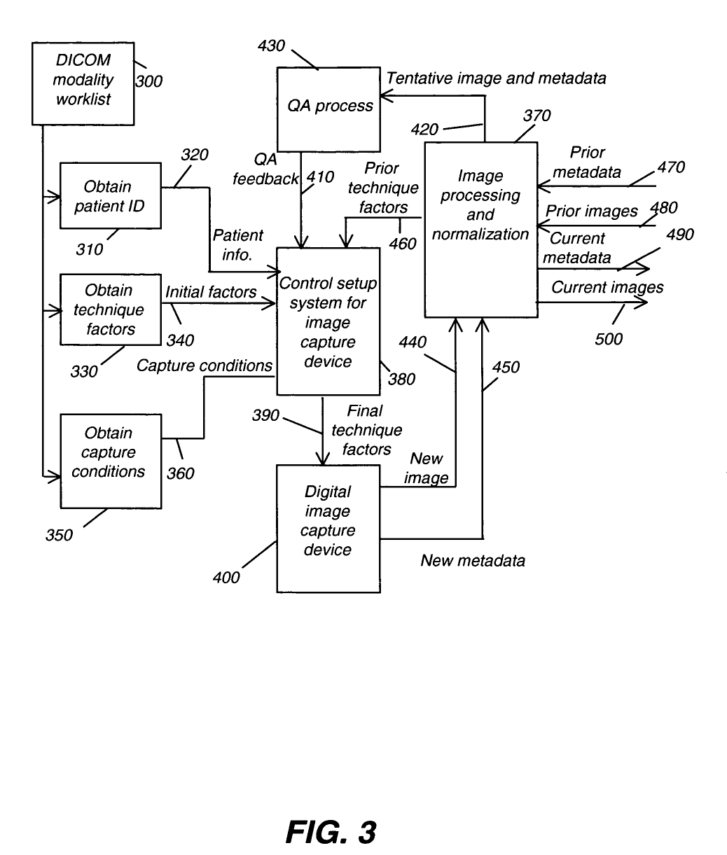

The block diagram of FIG. 3 shows the process whereby images are captured via digital radiography. Computed Radiography (CR) utilizes an x-ray sensitive storage phosphor sheet/plate, which is scanned and read by a separate reader device, yielding a digital image. Digital Radiography (DR) utilizes an x-ray sensitive sensor, which directly yields a digital image. This equipment, and the common application of these technologies, are well known to those skilled in the medical imaging arts.

Facilities that provide intensive care may have an RIS system or some equivalent system to manage the different radiology modalities. The initial setup for the capture of the radiograph begins in with a request that is in the form of a DICOM Modality Worklist 300, directing the technologist to capture a specific view of a specific patient.

DICOM Modality Worklist 300 includes patient information 320, and the RIS system provides an obtain patient information method 310. The policies and practices of the hospital facility, as well as the IT systems involved, provide the technologist with an obtain technique factors method 330. In this context, the technique factors are referred to as initial factors 340. These initial factors are specific to the case and area of interest.

Technique factors can include imaging metadata, with parameters such as the following:
(i) x-ray energy established via the peak kilovoltage (kVp) of the x-ray generator, indicative of the dosage level;
(ii) total filtration of the x-ray beam;
(iii) current setting in milliamperes (mA), establishing the intensity of the x-ray beam;
(iv) time of the exposure, expressed in seconds, indicative of the dosage level; and
(v) x-ray source to object (patient) distance.

In addition to exposure technique factors, it is of interest to acquire additional data about the image capture itself, using an obtain capture conditions method 350. These capture conditions 360 include, but are not limited to, the following imaging metadata parameters:
(i) exam type;
(ii) patient spatial orientation coordinates relative to any or all of the following: gravity, the imaging receptor, or the incident x-ray beam;
(iii) grid use and characteristics of grid;
(iv) patient size: including thickness and height dimensions, weight.

Using this approach, methods to capture both technique factors and capture conditions parameters can be integrated into a mobile X-ray/CR system (or DR system), as well as into an “intelligent bucky” sensing device, an imaging array used to obtain the digital image. As one example, integrated CR systems allow for technologist control settings to be captured directly. For another example, an “intelligent bucky” device integrated into a CR or DR system provides capabilities to record actual technique information used to obtain the image, such as imaging receptor orientation relative to the primary x-ray beam, grid usage and characteristics, and patient orientation relative to gravity.

Regardless of the method of capturing this image-related information, there is a need for a radiology technologist to accumulate this information and make the information available to the control setup system for an image capture device 380.

For the first image in the sequence of captured images, taken at a time t1, it is understood that there is no existing information concerning prior captures. For subsequent captures, taken at later times t2, t3, . . . tn, a feedback loop is used for the purpose of making subsequent image captures more like prior captures. The information required for this purpose, prior technique factors 460, is used to modify both the behavior of the technologist, as well as adjust the operation of the hardware/software parts of the image capture system.

The data set shown as final technique factors 390 is used to set up digital image capture device 400 and, subsequently, to capture the image. Digital image capture device 400 performs image processing, and yields a newly captured image (new image 440) and its corresponding metadata (new metadata 450). New image 440 is sometimes referred to as a “raw” image. These are delivered to an image processing and normalization process 370, where the image is processed for viewing, with regard to the type of capture device, and the procedure of interest.

Image processing and normalization process 370 makes use of prior metadata 470 and prior images 480, as well as new image 440 and new metadata 450. New metadata 450 includes final technique factors 390 corresponding to all prior images of the same patient of the same exam type, and radiologist reports from the prior exams, if these reports exist. The purpose of capturing and maintaining the new metadata is to improve subsequent image data capture consistency, improve the ability to process the image for display or presentation and visual interpretation in the most consistent way, and improve subsequent data analysis, for example, by means of ICU CAD process 110, as described in more detail subsequently. In this method, comparisons are made between or among chronologically sequential images, and inferences or conclusions drawn based on these comparisons, as described subsequently.

A quality assurance feedback loop 410 involving the technologist is described as a quality assurance process 430. The newly captured image and its metadata, referred to as tentative image and metadata 420, are presented to the technologist. Quality assurance work involves such activities as assuring that the systems functioned properly and obtained the correct image, that the patient was properly positioned, and that suitable windowing and leveling for viewing are applied per procedure. When the image capture procedure is acceptable to the technologist, the system produces current images 500 and current metadata 490. Additional processing can then be performed to integrate current metadata 490 and images 500 with prior metadata 470 and prior images 480 in anticipation of the next imaging session for the patient.

Note that the image that is captured can be a simple projection radiograph. However, the method and apparatus of the present invention can be used with any of a number of other imaging modalities, as well as where multiple imaging modalities apply for a single patient.

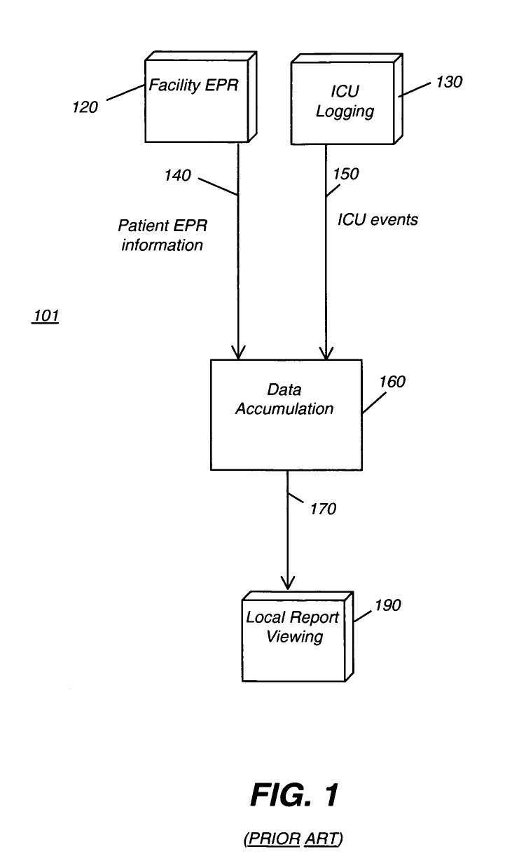

As described above with reference to FIGS. 1-3, management of medical images for an ICU patient to support longitudinal tracking has the steps of obtaining a first set of images at a time t1, taken under a set of imaging parameters, and storing the set of imaging parameters as part of prior metadata; then obtaining a second set of images at a time t2 according to at least some portion of the set of imaging parameters used for the first set of images. Measurement data, stored locally in the ICU or available from the facility EPR system 120, can then be obtained for the patient, including personal medical data and instrumentation measurements stored for the patient. Processing the second set of images can then be executed, using data obtained from the first set of images and using the measured data to obtain a set of diagnostic results. An appropriate operator instruction enables patient data and the set of diagnostic results to be displayed for the clinician, as is described subsequently. This process can be extended beyond first and second sets of images to include any number of additional sets of images where useful. This same process can also be used with non-image data such as measurement data for a patient.

ICU CAD process 110 provides computer assisted interpretation of each individual ICU image, and computer assisted interpretation of each ICU image relative to one or more corresponding prior images of the same patient, also referred to as change analysis. By means of proprietary algorithms, utilizing raw images from various digital image capture devices, and using image metadata including technique factors and patient information, the system is capable of detecting some specific features of digital x-ray images that can be of particular relevance to an ICU patient. At least two basic types of features can be detected. The first include the anatomical placement of portions of feeding and breathing tubes, such as tips, and of tips and other portions of catheters and PICC (Peripherally Inserted Central Catheter) lines. The second group of features of interest include characteristics of various disease processes.

The relative anatomical placement of tips and tubes is of particular interest. This particular aspect of the computer-assisted interpretation is of interest because of potentially severe results when tubing is misdirected and the tips are misplaced. The use of EPR and ICU Log data, as well as capture conditions, is significant because the information provided aids in the interpretation of the image data. As one example, the detection of the anatomical placement of a feeding tube and its tip, as noted using EPR data and/or ICU log information for a given chest X-ray and a given procedure, has a higher probability of success than does a simple visual examination of the image.

ICU CAD process 110 also provides computer assistance in the detection and analysis of various disease processes and features corresponding to patient conditions or changes in the patient condition. Examples include pneumothorax detection, assessment of changes in fluid levels relative to the degree of inspiration, and heart size assessment. Conditions such as these can be detected by comparing images obtained at different times t1, t2 using CAD utilities known to those skilled in the diagnostic imaging arts.

It is noted that the condition of a patient in the ICU can change continuously. However, the progression of change for any condition, over time, can be subtle. It is of interest, therefore, to have images that are captured consistently, over time, as part of the detection process. Utilizing prior capture conditions, technologist feedback, actual technique factors for image capture, and knowledge of the patient disease condition, along with stored measurement data from the EPR and/or ICU Log data, the system is capable of generating images which share a more consistent rendering and are taken from the same perspective and under the same conditions. This allows the clinician or radiologist a better opportunity to visually detect differences, and improves the likelihood that ICU CAD algorithms can detect very subtle differences. The additional measurement data enhances the richness of information that is now available, giving the clinician a full battery of data with which to diagnose the patient and provide beneficial treatment.

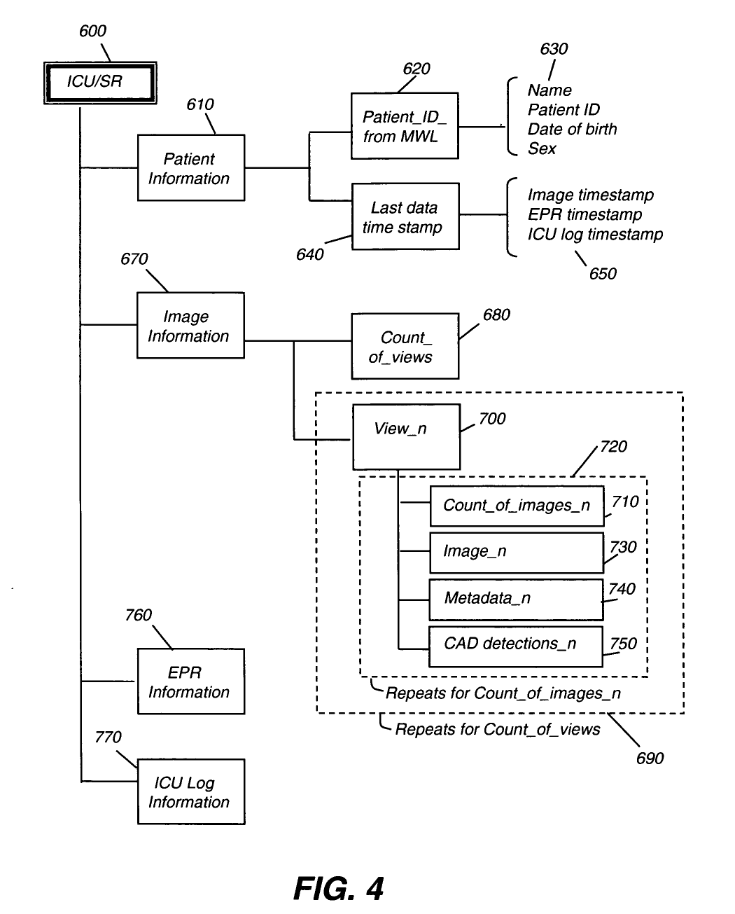

The block diagram of FIG. 4 shows information that can be obtained by data accumulator 160 (FIG. 2) for a given patient. This data is represented spatially as it could be organized in the form of an Intensive Care Unit Structured Record (ICU/SR) 600. The DICOM Structured Record (SR) is a construct well known to those skilled in the medical information processing arts. Use of this data structure helps to foster traceability, verifiability, and completeness of data, while minimizing storage redundancy. It also provides data in a standard format, allowing the stored data to be accessible to other DICOM-conformant systems.

The use of the Structured Report (SR) as implemented in this embodiment of the present invention is two-fold. First, its use makes explicit the relationships between the data presented and the conclusion drawn. Secondly, its use allows for the ICU/SR to become part of the formal record of the medical facility. In the first case, it is important that the relationship between data presented and conclusion drawn is explicit. In the case of computer aided detection systems, for example, providing that explicit link is useful to the clinician and the radiologist who looks at the data provided by the SR, because it improves confidence in the system and provides these specialists with a reasonable chain of conclusion well suited to their accepted practices. It is also useful in improving communications between and among all clinically involved parties because it offers a rich information source. In the second case, the SR has characteristics which allow the record to become a formal part of the care facility's documentation trail.

As is known to those skilled in the medical information arts, the content nor management information that are encoded in a structured report (SR) can be altered without creating a new instance. Amendments or revisions to SR data generate new document instances. Specific rules govern relationships such as references between duplicate data and between different versions of an SR. For example, identical documents and prior versions of a document are referenced to the most recent document. Status and verification encoding is also provided for DICOM SRs, allowing traceability to the latest and most complete SR where there are multiple versions and providing verification by individuals responsible for report content.

The structure of the SR data has some correspondence to radiology procedures of the hospital facility. Where conventional workflow provides orderly scheduling, execution, and archival of images for the radiologist or other clinician, the system and method of the present invention adds the features of ICU CAD processing and the benefits of CAD interaction with EPR and ICU log data, with the added benefits of providing data in a chronological sequence to simplify viewing and facilitate diagnosis.

Of particular interest is the existence of data records, which describe some measured or observed data from the patient. One of the aspects of the present invention is to allow the ICU clinician, and/or other interested parties, to see recorded events that have occurred, in chronological sequence, all in one presentation, so that a determination of progress and effectiveness of treatment may be made. This promotes improved communication between the clinician in the ICU and a radiologist or other specialist, on site or at another location. The available relevant information is accessible by means of this single container of information and can be kept as current as ICU and IT policies and practices prescribe.

There can be a number of discrete collections of information accumulated in the system. FIG. 4 shows the overall structure of an Intensive Care Unit Structured Record ICU/SR according to one embodiment. Here, the collected data for an ICU patient is organized under the following information groupings:

(a) Patient information 610;

(b) Image information 670;

(c) EPR information 760; and

(d) ICU log information 770.

The information accumulated for the ICU patient and accessible to the system can then be associated with the relevant date and time of the observation.

In the embodiment shown, patient information 610 is arranged in two parts:
(i) Patient_ID_from_MWL 620, where MWL stands for Modality Worklist consists of data such as the patient name, in-facility patient ID, date of birth, and sex, referred to as patient ID detail 630.
(ii) Last data time stamp 640 stores information recording the last time data is entered into the record, via the Image Timestamp, EPR Timestamp, and ICU Log Timestamp, collectively stored as time stamp detail 650.

Image information 670 includes the captured images, metadata, and computer aided diagnostic information, generated by the system. One data element within this section, count_of_views 680, represents the number of different sequences of images for the associated patient.

A typical case may only have one procedure generating one view, repeated over a regular time interval. An ICU can expect to have patients who require a series of images, generating more than one view, or patients who may require multiple, sequential imaging studies. To accommodate these cases, there is a collection of data whose section structure repeats for count_of_views 680 as indicated by a repeat box 690. For each view_n 700, there is a data element within this section, count_of_images_n 710, which represents the number of separate and distinct image items within the sequence.

Within image information 670 is a collection of data whose section structure repeats for count_of_images_n, as indicated by a repeat box 720. Repeated elements are image_n 730, metadata_n 740, and CAD detections_n 750.

A DICOM-compliant file containing an image also contains a significant amount of image metadata, such as additional data about the image including conditions under which it was obtained. Depending upon the specific implementation of the imaging modality, the file could be generated to contain any amount of metadata, including image-specific metadata or even all available patient metadata. In one embodiment, the accumulated data within image_n 730 and metadata_n 740 represents all that is known about the setup and conditions used for the image capture. Again, it is noted that the image data and associated image metadata may be x-ray or any other suitable type of patient image.

CAD detections_n 750 stores the output of ICU CAD process 110 (FIG. 2) for a specific image or sequence of images. For example, data elements stored in CAD detections_n 750 may include the following:
(i) Operating parameters set via either policy/practice or ad hoc use;
(ii) Indicators that tube/line or tube/line tip locations were discovered;
(iii) Vectors describing tube/line locations within an image, if needed;
(iv) Vectors describing tube/line tip locations within an image, if needed; and
(v) Vectors describing the location of the relevant anatomy within an image.

EPR Information 760 is a local repository for information obtained from, or information referred to within, the electronic patient record system of the ICU facility. EPR systems within health care facilities range from paper-based systems to extremely sophisticated data management systems. Health care facilities with EPR systems capability populate those systems with various data, including, but certainly not limited to: demographic information, facility history with patient, patient history, pharmaceutical drug related, diagnostic lab results related, medical condition histories, diagnosis history, current treatments, and current health care payer information. Depending upon the sophistication of the facility's IT EPR systems, relevant EPR data may or may not be accessible to EPR system communications by means of ICU CAD process 110. If this data is available via intersystem communications, facility IT policy may require that only references to, rather than copies of, EPR data be retained for subsequent use. Regardless, EPR information required to be available and useful to ICU CAD process 110 should be available to the ICU CAD system.

ICU log information 770 stores data accumulated ‘locally’ within the ICU, which may or may not be part of the facility's IT EPR system. In a fully integrated IT environment, the data referenced in ICU log information 770 would be stored by the facility EPR system; in such a case ICU log information 770 may simply store a null set or a reference to the data that is stored elsewhere.

Chronologically Arranged Information Sets are now described.

One capability of the system of the present invention relates to an improved capability for making patient history available to the clinician and allowing a number of options for display and use of stored patient information. In particular, images and information about the patient can be requested from the system. Results of test data and images taken at different times can then be available to the clinician, presented in any of a number of preferred formats.

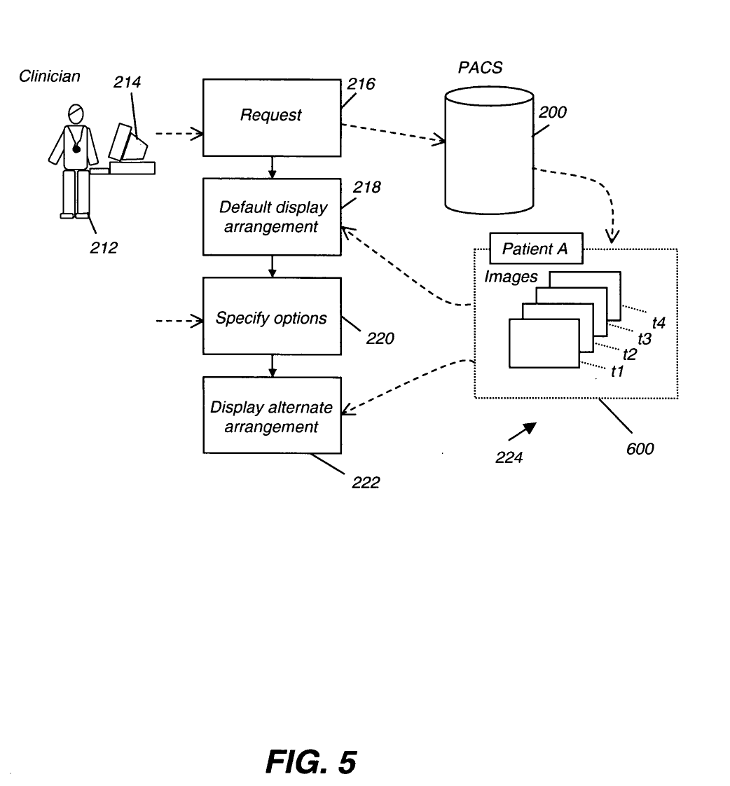

Referring to FIG. 5, there is shown a block diagram with the basic processes and data that provide the capability for obtaining a chronologically arranged information set according to one embodiment. A clinician 212 at a workstation 214 enters a request 216 for image sequences and other historical information relating to a patient. Request 216 goes to PACS system 200 in the form of a DICOM worklist. The PACS system responds by providing image and other data stored for the patient represented generally as patient data 224 and typically provided in the form of a structured record (SR), such as ICU/SR 600 or similar record as shown in FIG. 5. As was described with reference to FIG. 4, patient data 224 can include, for example, image data taken at different times t1, t2, t3, . . . tn. The PACS system provides a default display arrangement 218 that specifies an image presentation format and layout in a standard format. Clinician 212 can enter specific options, with an options instruction 220 for alternate arrangements of displayed images and data, typically using a predetermined format. For example, for lung imaging, a standard radiologist-preferred arrangement or “hanging protocol” showing different views in a certain layout order may used as the default. However, an alternate hanging protocol may be preferred by an individual radiologist or for certain types of cases. In response to a command from clinician 212, a display alternate arrangement process 222 is executed. This command may simply be entered using conventional windowing management utilities, using a mouse or other pointer, with techniques generally familiar to users of personal computers.

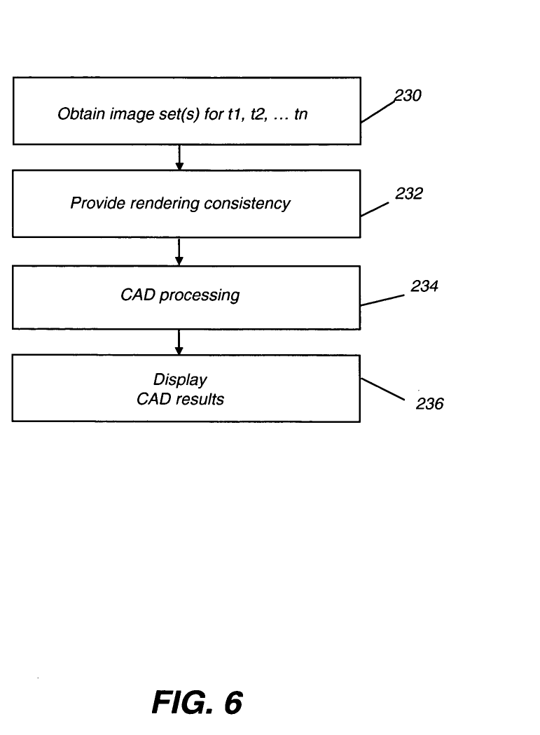

FIG. 6 is a block diagram showing key steps of the overall process for displaying chronologically arranged image data, as carried out by the system of the present invention. In an obtain image sets step 230, the system obtains from the PACS system multiple image sets taken at different times t1, t2, . . . tn. A rendering consistency step 232 uses image processing utilities for consistent presentation of images that may be taken at different times, but are of substantially the same body tissue, in a consistent manner. Thus, for example, two lung images taken on different days or under slightly different conditions may exhibit different contrast ranges. Consistent rendering utilities attempt to adjust the contrast of one or more images in order to allow them to be comparable with the presentation of images taken at different times. Other algorithms may attempt to register images to each other in order to allow a quick comparison of images taken over the same tissue area minutes, hours, or days earlier. This recognizes the difficulty of obtaining images that would be exactly aligned; instead, it is sufficient that two images cover substantially the same body tissue. Rendering consistency step 232 typically employs technique factors obtained at the time the images were captured, as was described with reference to FIG. 3. Other methods used in rendering consistency step 232 may use information obtained from the images themselves, such as identification of background range and density range over Regions of Interest.

An optional CAD processing step 234 may be executed in order to run various CAD algorithms on any of the images obtained for the patient. One advantage of this arrangement is that CAD algorithm results can be compared and the results of this comparison provided to help identify a problem area within an identified Region Of Interest (ROI). That is, for two or more images obtained from substantially the same body tissue but taken at different times, CAD algorithm results can be compared to highlight particular problem areas to the clinician, including rapidly developing conditions. In one embodiment, CAD processing is performed on two or more images, each image having been taken at a separate time t1 or t2, respectively. In comparing CAD results, an abrupt change in characteristics of a portion of tissue may help to highlight progress of a disease condition or treatment. Such an abrupt change, for example, may be reported by positioning a marker on a displayed image or using some other mechanism that is commonly employed by CAD systems.

It is noted that earlier CAD results can be saved, stored as shown in the example data structure of FIG. 4, but need not be saved, particularly where they do not show information of particular interest. CAD routines can be re-run on earlier as well as on later images, allowing a particularly useful tool for assessing growth rate or eliminating dormant or benign tissue from consideration. A CAD results display step 236 then follows the optional CAD processing step 234, again with the option for running CAD algorithms on previous images.

Analogous steps to those used for image arrangement can also be used for presenting other types of data or measurements about the patient that were obtained at different times. For example, blood test values taken at various intervals may be displayed numerically or graphically in order to allow the clinician to more readily spot a trend or watch fluctuations in a vital measurement that may indicate the need for preventive intervention.

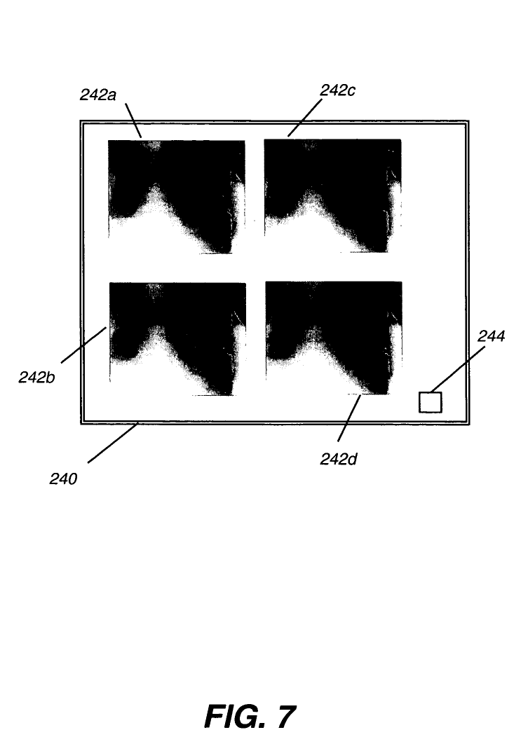

The plan view of FIG. 7 shows an example display 240 with the option of chronologically arranged data displayed for the clinician. Images 242a, 242b, 242c, and 242d, obtained at a time t1 for this patient, are stored on the PACS system. An icon 244 on display 240 enables selection of images of the same view from an earlier imaging session, t2. Other controls and commands could be provided to initiate CAD operation for a particular image or to flag an area of interest on one or more images for further analysis.

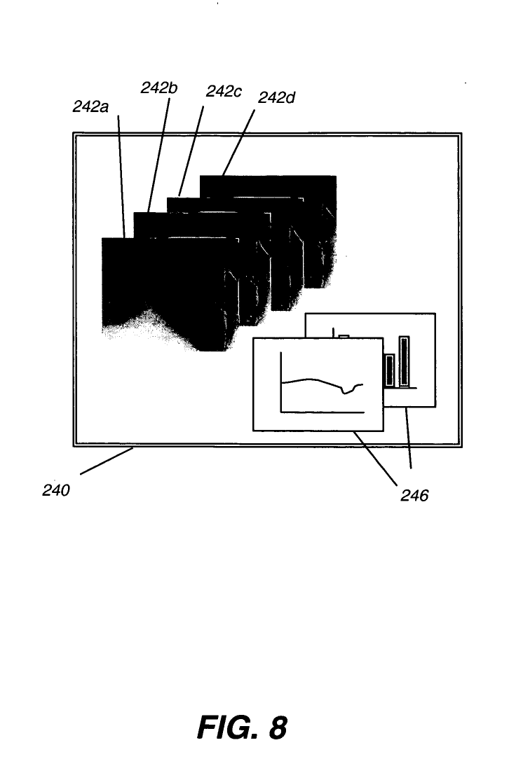

In the plan view of FIG. 8, alternative display arrangements for images and measured data obtained at different times t1, t2, . . . tn are shown. Here, for example, images 242a, 242b, 242c, and 242d have been obtained from substantially the same body tissue, but are captured at different times, and are arranged on display 240 using staggered windows, following the well-known windowing scheme familiar to personal computer users. This allows the clinician to use standard window selection, positioning, and sizing tools for obtaining a larger view of any individual image or for placing two images 242a, 242b, 242c, or 242d side by side, for example. Graphs 246 provide a useful method for evaluating changes in measured data taken at different time periods. Graphs 246 or tabular data giving vital measurement data, presented using the windowing data presentation paradigm, can also be sized, positioned, and otherwise manipulated on a display monitor to suit the viewing clinician.

The methods and apparatus of the present invention can help to provide improved care in an ICU or other type of critical care facility. Particularly well suited to support longitudinal tracking, the methods of the present invention provide imaging and other data in a chronologically sequenced arrangement, helping the clinician to be alerted to changes in the condition of a patient that can be detected using image and measured data. The present invention helps to take advantage of different sources of data so that information can be provided to medical personnel in a form that is straightforward to understand and use.

The invention has been described in detail with particular reference to certain preferred embodiments thereof, but it will be understood that variations and modifications can be effected within the scope of the invention as described above, and as noted in the appended claims, by a person of ordinary skill in the art without departing from the scope of the invention. Patient images, data, and metadata can be provided to the clinician in a number of ways within the scope of the present invention. Patient images could be of different types or modalities, including x-ray or ultrasound images. For example, various other data components could be used in ICU/SR 600 for storing patient metadata and image information. A variety of different types of computer hardware and networked computer platforms could be employed in order to implement ICU patient data information system 101 as described with reference to FIG. 2. Individual functions such as image processing process 60, ICU CAD process 110, and data accumulator 160 could be performed by a single computer, by dedicated workstations, or could be functions distributed along a network.

Thus, what is provided is an apparatus and method for providing clinical imaging information for a patient in an intensive care unit.

### Parts List

10 Image capture setup instruction
11 Image capture section
20 Initial conditions
30 Digital image capture process
40 Prior conditions
50 New capture
60 Image processing process
70 Prior information
80 Current information
90 Images
100 Metadata
101 Information system
105 CAD detections
110 ICU CAD process
111 ICU CAD section
120 EPR system
130 Logging apparatus
131 Data accumulation section
140 EPR information
150 ICU events
160 Data accumulator
170 Data
180 ICU/SR
190 Report viewing station
200 PACS/RIS system
201 External reporting section
210 RIS response
212 Clinician
214 Workstation
216 Request
218 Default display arrangement
220 Specify options instruction
222 Display alternate arrangement process
224 Patient data
230 Obtain image sets step
232 Rendering consistency step
234 CAD processing step
236 CAD results display step
240 Display
242a, 242b, 242c, 242d Images
244 Icon
246 Graph
300 DICOM Modality Worklist
310 Obtain patient information method
320 Patient information
330 Obtain technique factors method
340 Initial factor
350 Obtain capture conditions method
360 Capture conditions
370 Image processing and normalization process
380 Image capture device
390 Final technique factor
400 Digital image capture device
410 Quality assurance feedback loop
420 Tentative image and metadata
430 Quality assurance process
440 Image
450 Metadata
460 Prior technique factor
470 Prior metadata
480 Prior images
490 Current metadata
500 Current images
600 Intensive Care Unit Structured Record (ICU/SR)
610 Patient Information
620 Patient ID from MWL
630 Patient ID detail
640 Last data time stamp
650 Time stamp detail
670 Image information
680 Count of views
690 Repeat box
700 View_n
710 Count of images_n
720 Repeat box
730 Image_n
740 Metadata_n
750 CAD detections_n
760 EPR information
770 ICU log information

1. A method for tracking of a patient in a critical care facility, the method comprising the steps of:
a) obtaining a first diagnostic image at a time t1, taken using a first set of imaging parameters;
b) storing at least a portion of the first set of imaging parameters;
c) obtaining a second diagnostic image at a time t2, time t2 being later than time t1, taken using a second set of imaging parameters, wherein the first and second diagnostic images are of substantially the same body tissue;
d) storing at least a portion of the second set of imaging parameters;
e) identifying a region of interest from either the first or second diagnostic image;
f) executing a computer aided diagnostic process for a portion of the region of interest for each of the first and second diagnostic images to generate first and second image results; and
g) comparing the first and second image results.

2. The method of claim 1 wherein obtaining a first diagnostic image comprises obtaining an x-ray image.

3. The method of claim 1 wherein identifying a region of interest further comprises running a computer-aided diagnostic process.

4. The method of claim 1 further comprising obtaining measurement data stored for the patient.

5. The method of claim 1 wherein using the second set of parameters comprises using the first set of imaging parameters.

6. The method of claim 1 further comprising registering the first and second diagnostic images to each other to provide spatially registered diagnostic images.

7. The method of claim 1 further comprising normalizing the first and second diagnostic images to improve consistency of the display.

8. A method for managing medical images for a patient in a critical care facility, the method comprising the steps of:
a) obtaining a first diagnostic image at a time t1, taken using a first set of imaging parameters;
b) storing at least a portion of the first set of imaging parameters;
c) obtaining a second diagnostic image at a time t2, later than time t1, taken using a second set of imaging parameters, wherein the first and second diagnostic images are of substantially the same body tissue;
d) storing at least a portion of the second set of imaging parameters; and
e) providing a display displaying at least the first diagnostic image and the second diagnostic image, arranged in chronological order on a display monitor.

9. The method of claim 8 wherein obtaining a first diagnostic image comprises obtaining an x-ray image.

10. The method of claim 8 further comprising the steps of:
obtaining measurement data from the patient at a time t3 and at a later time t4; and
providing a graphical display showing measurement data values for at least times t3 and t4.

11. The method of claim 8 wherein obtaining the second diagnostic image comprises obtaining at least a portion of the first set of imaging parameters, in response to a worklist instruction

12. The method of claim 8 further comprising:
f) identifying a region of interest from either the first or second diagnostic image;
g) executing a computer aided diagnostic process for a portion of the region of interest on each of the first and second diagnostic images; and
h) comparing results of the computer aided diagnostic process from both first and second diagnostic images.

13. A system for management of patient images in a critical care facility comprising:
a) a data accumulator in communication with at least one storage device within the care facility for obtaining data about the patient therefrom;
b) an image processor in communication with the data accumulator for obtaining information on imaging conditions for images previously obtained and for obtaining digital images of the patient according to the imaging conditions;
c) a CAD processor in communication with the image processor for obtaining imaging metadata and images and providing diagnostic results to the data accumulator;
d) a display monitor in communication with the data accumulator for display of CAD processor results; and
e) a PACS/RIS system in communication with the data accumulator for accepting report data from the data accumulator.

14. The system of claim 13 wherein the digital images are taken from the group consisting of digital x-ray images, CAT scan images, and ultrasound images.

15. A method for managing medical images for a patient in a critical care facility, the method comprising the steps of:
a) obtaining a first diagnostic image at a time t1, taken using a first set of imaging parameters, and storing at least a portion of the first set of imaging parameters;
b) obtaining a second diagnostic image at a time t2, later than time t1, taken using a second set of imaging parameters, and storing at least a portion of the second set of imaging parameters; and
c) forming a data structure comprising the first and second diagnostic images and further comprising information about the patient.

16. The method according to claim 15 further comprising normalizing the first and second diagnostic images to improve presentation consistency.
